(19) United States
(12) Patent Application Publication
(10) Pub. No.: US 2005/0096543 A1
(43) Pub. Date: May 5, 2005

(54) Motion tracking for medical imaging

(76) Inventors: John I. Jackson, Menlo Park, CA (US); Liexiang Fan, Issaquah, WA (US); Matthew M. Holladay, Britton, MI (US); David E. Gustafson, North Bend, WA (US)

Correspondence Address: Siemens Corporation;Attn: Elsa Keller, Legal Administrator, Intellectual Property Department, 170 Wood Avenue South, Iselin, NJ, 08830, US

(21) Appl. No.: 10/861,268

(22) Filed: Jun. 4, 2004

Related U.S. Application Data
(60) Provisional application No. 60/516,778, filed on Nov. 3, 2003.

(51) Int. Cl. A61B 8/00

(52) U.S. Cl. 600/441; 600/453; 600/449

(57) ABSTRACT

Motion of a region of interest is tracked in medical imaging. For example, velocity information, such as colored Doppler velocity estimates independent of tracking from one image to another image, are used to indicate an amount of motion between the images. The velocity assisted tracking may be used for one dimensional tracking (e.g., for M-mode images), for tracking in two dimensional images, or for tracking between three dimensional representations. As an alternative or additional example, physiological signal information is used to assist in the tracking determination. A physiological signal may be used to model the likely movement of an organ being imaged to control or adjust matching search patterns and limits. The modeling may also be used to independently determine movement or for tracking. The independently determined tracking is then combined with tracking based on medical data or other techniques. The cost function or other metric for determining the sufficiency or a match may include information modeled from or selected as a function of the physiological cycle signal in addition to other matching calculations. The fusion of physiological signal information with image data tracking may improve the tracking.

### REFERENCE TO RELATED APPLICATIONS

The present patent document claims the benefit of the filing date under 35 U.S.C. §119(e) of Provisional U.S. Patent Application Ser. No. 60/516,778, filed Nov. 3, 2003, which is hereby incorporated by reference.

### BACKGROUND

The present invention relates to motion tracking for medical imaging. In particular, methods and systems for determining the position of a region of interest from one image within a second image are provided.

The region of interest of one image is matched with another image to determine the movement or change in location of an object for a sequence of images. Various motion tracking algorithms have been used. For example, a region of interest of one image is correlated with a different image to identify a best or sufficient match indicating a translation and/or rotation between images. As an alternative to correlation, a sum of the square of the differences or other cost function identifies the sufficiency of a match.

U.S. Pat. No. 6,193,660, the disclosure of which is incorporated herein by reference, discloses methods and systems for quantitative analysis of one or more regions within a series of ultrasound images where the regions move between images. Correlation is used to track the movement of the region. U.S. Pat. No. 6,201,900, the disclosure of which is incorporated herein by reference, identifies a change in position of a region in order to register the images for forming an extended field of view or a three dimensional reconstruction of a volume. By tracking the motion or region of interest between subsequent images, the relative positioning of the images with respect to each other is determined. U.S. Pat. No. 6,527,717, the disclosure of which is incorporated by reference herein, tracks changes in position between subsequent images to calculate two dimensional velocity vectors. For example, data correlation or a motion sensor on a transducer determines an amount of motion associated with a region between images. The amount of motion is then used to alter or calculate an actual represented velocity associated with one image. The corrected velocity values or estimates are used to calculate strain or strain rate.

Various sources of motion contribute to difficulties in tracking a region of interest between images acquired at different times. For example, an organ, such as the heart, has a unique motion that contributes to the motion of a region of interest for cardiac imaging in addition to any transducer or patient motion. Motion due to the breathing cycle may also contribute to inaccuracies in tracking. As an organ contracts or expands, a subsequent correlation may provide a less than ideal match.

### BRIEF SUMMARY

By way of introduction, the preferred embodiments described below include methods and systems for tracking motion of a region of interest in ultrasound imaging. For example, velocity information, such as colored Doppler velocity estimates independent of tracking from one image to another image, are used to indicate an amount of motion between the images. The velocity assisted tracking may be used for one dimensional tracking (e.g., for M-mode images), for tracking in two dimensional images, or for tracking between three dimensional representations. As an alternative or additional example, physiological signal information is used to assist in the tracking determination. A physiological signal may be used to model the likely movement of an organ being imaged to control or adjust matching search patterns and limits. The modeling may also be used to independently determine movement or for tracking. The independently determined tracking is then combined with tracking based on ultrasound data or other techniques. The cost function or other metric for determining the sufficiency of a match may include information modeled from or selected as a function of the physiological cycle signal in addition to other matching calculations. The fusion of physiological signal information with image data tracking may improve the tracking.

In a first aspect, a method is provided for tracking motion of a tissue object region of interest in medical imaging, such as ultrasound, computed tomography, magnetic resonance, positron emission or other medical imaging. The tissue object region of interest is identified in a first image. A velocity is estimated for at least one location associated with the tissue object region of interest. A position of the tissue object region of interest in a second image is determined as a function of the velocity. The first image is acquired at a different time then the second image.

In a second aspect, a system is provided for tracking motion of a tissue object region of interest in medical imaging. A display is operable to display a sequence of images. A memory is operable to store an indication of the tissue object region of interest in a first image of the sequence of images. A velocity estimate processor is operable to estimate a velocity for at least one location associated with the tissue object region of interest. A further processor is operable to determine a position of the tissue object region of interest in a second image of the sequence of images as a function of the velocity. The first image is acquired at a different time than the second image.

In a third aspect, a method is provided for tracking a region of interest in medical imaging. The region of interest is identified in a first image. A physiological cycle signal is obtained. A position of the region of interest in a second image is determined as a function of the physiological cycle signal. The second image is different then the first image.

In a fourth aspect, a method is provided for tracking a region of interest in medical imaging. A first estimate of a position of a region of interest in a first image relative to a second image is obtained as a function of a first type of data. A second estimate of the position of the region of interest in the first image relative to the second image is obtained as a function of a second type of data different then the first type of data. The position is then identified as a function of the first and second estimates.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of an ultrasound system for motion tracking;

FIG. 2 is a flow chart diagram of one embodiment of a method for tracking motion of a region of interest;

FIG. 3 is a graphical representation of one embodiment of an M-mode image with a tracked region;

FIG. 4A is a flow chart diagram of one embodiment of using a physiological signal to assist in motion tracking;

FIG. 4B is a flow chart diagram of another embodiment of using a physiological signal to assist in motion tracking;

FIG. 5 is a flow chart diagram of one embodiment of a method for generating motion model related landmark data;

FIG. 6 is a flow chart diagram of another embodiment for limiting or assisting searches as a function of physiological signal information; and

FIG. 7 is a flow chart diagram representing one embodiment for obtaining information indicating a change in position between images.

### DETAILED DESCRIPTION OF THE DRAWINGS AND PRESENTLY PREFERRED EMBODIMENTS

Tracking of a region of interest is performed between images using velocity of an object in one embodiment. In another embodiment, physiological information is used to assist and/or determine the position of a region of interest in different images. In yet other embodiments, both the physiological cycle signal information and velocity of the object information are used to assist or determine the position of a region of interest in a plurality of different images.

FIG. 1 shows one embodiment of a system 10 for tracking motion of a region of interest in ultrasound imaging. The system includes a B-mode detector 12, a velocity processor 14, a memory 16, a processor 18, a display 20, and a user interface 22. Additional, different or fewer components may be provided, such as providing the system 10 without the B-mode detector 12, without the user interface 22, with scan converters, with other types of detectors or with other now known or later developed diagnostic ultrasound imaging system components. The system 10 is a handheld, portable, cart mounted or permanent ultrasound system with transmit and receive beamformers for obtaining ultrasound data with a transducer. In other embodiments, the system 10 is a work station free of beamformers. In yet other embodiments, the system 10 is a computed tomography, magnetic resonance, x-ray, positron emission or other medical imaging system.

The B-mode detector 12 receives ultrasound data representing different spatial locations along a signal scan line, such as for M-mode imaging, or along a plurality of scan lines, such as for two dimensional or three dimensional imaging. The intensity, power, magnitude or other characteristic of the received data is detected for the spatial locations. Each detected frame of data represents a scanned region at a given time or time range, such as a one dimensional region for M-mode imaging or a multi-dimensional region for two or three dimensional imaging. By repetitively acquiring and detecting data within the scan region of the patient, a sequence of images is generated. The images are stored in the memory 16 and/or provided directly to the display 20. In alternative embodiments, the images include additional information, such as flow information from the velocity processor 14. Contrast agent, harmonic or other detection techniques using the B-mode detector 12, the velocity processor 14, or other detection components may alternatively or additionally be used for forming a sequence of images. For example, a combination B-mode and flow mode images are generated for each of a sequence of images. Images from other modalities of medical imaging are acquired with any known or later developed detector.

The display 20 is a monitor, LCD, CRT, plasma screen, projector, or other now known or later developed display device. The display 20 is operable to display the sequence of images. For example, the display 20 displays an M-mode image formed from the scan of a one dimensional scan line as a function of time. Temporally different portions of the M-mode image represent different M-mode images within a sequence used to form the entire two dimensional M-mode representation. As another example, the sequence of images includes a plurality of two dimensional B-mode or other types of detected images. As yet another example, the sequence of images includes a plurality of two dimensional Doppler images. In another example, a plurality of three dimensional representations of a volume is provided. Combinations of these different types of imaging may also be used.

Any of various patient regions or volumes may be scanned. For example, apical views of the heart are ultrasonically scanned. Two chamber, four chamber, long axis, or other apical views may be used. As yet other examples, arteries, veins, organs, muscle, or other patient regions are scanned.

The displayed images represent the scanned region of a patient at a generally given time. The sequence of images represents the scanned region over time. The images indicate the region relative to the transducer. For example, the display is operable to indicate the position of a tissue object region of interest in each of the sequence of images. A tissue object region of interest corresponds to biological tissue as opposed to fluids, such as blood. For example, a tissue object region of interest is a region of interest including heart muscle tissue with or without associated blood pool regions adjacent to the tissue object. In alternative embodiments, a region of interest corresponds to a fluid, fluid area, or both tissues and fluid.

The memory 16 is a RAM, ROM, hard drive, removable media, CD, disk, buffer, combinations thereof, or other now known or later developed memory device. In one embodiment, the memory 16 is configured as CINE memory, but other memory configurations may be used. The memory 16 is operable to store the sequence of images for non-real time generation of one or more images of the sequence on the display 20. The memory 16 may alternatively provide a pipeline for passing images to the display 20 for real-time imaging with an ultrasound system. The memory 16 or a separate memory associated with the processor 18 or other component of the system is operable to store an identification of a region of interest in one or more of the images of the sequence of images. For example, the memory 16 stores spatial coordinates associated with a user or processor determined tissue object region of interest for an image or a plurality of images.

The velocity processor 14 is an application specific integrated circuit, general processor, digital signal processor, Doppler processor, flow estimator, correlator, digital circuit, analog circuit, combinations thereof or other now known or later developed device for estimating a velocity associated with a scanned object, tissue, or fluid. For example, two or more sequential pulses are transmitted and received along a same or similar scan lines. The change in phase or the first lag of the auto correlation between the received data from the multiple transmissions is used to estimate the velocity of the scanned spatial locations. The multiple transmissions and receptions are used to form velocity estimates for a given frame of data or represent a generally given time, such as a time corresponding to a B-mode image acquisition. The generally given time includes time to interleave B-mode and Doppler pulses for generating a combination B-mode and Doppler image. The scan lines used for velocity estimation may be the same or different than for other imaging. In one embodiment, the velocity estimate processor is operable to estimate a velocity for at least one location associated with a region of interest, such as a tissue object region of interest. For example, the velocity processor 14 includes a clutter filter for identifying velocities associated with tissue movement or velocities associated with fluid movement. Velocities of fluid or tissue are estimated separately for each image or frame of data.

In other medical imaging modalities, the velocity processor 14 is an application specific integrated circuit, general processor, digital signal processor, flow estimator, correlator, digital circuit, analog circuit, combinations thereof or other now known or later developed device for estimating a velocity associated with a scanned object, tissue, or fluid. For example, in CT or digital X-ray imaging, tissues with different x-ray absorption properties, such as interfaces between air in bronchial tree or alveoli and parenchyma, or the plural surface of lobes of the lung, are detected which allows these structures to be tracked and the velocity of these moving tissues to be calculated such that compensation for respiratory motion can be accomplished. By the injection of x-ray contrast material into flowing blood, it is also possible to identify contrast filled vessels, allowing for example, contrast filled coronary artery velocities to be calculated during high frame rate cardiac angiographic procedures. Similar processing can be accomplished in magnetic resonance imaging (MRI), wherein free-induction decay signals are detected, most typically for protons (H+), as spins are realigned with the static magnetic field. MRI also may use a more complicated RF excitation spin-selection method, such as used in cardiovascular MR applications to track myocardial tissue, wherein spins are intentionally realigned in such a way as to generate a “grid” which is seen in the MRI images allowing “tagged” tissue regions to be tracked over the cardiac cycle. Alternatively, finite element modeling with any number of landmarks may be used to determine the velocity. One or more images are used to determine the velocity associated with structure (tissue or fluid) of a particular image. The velocity is used to determine a region of interest position in yet another image.

The processor 18 is a control processor, general processor, digital signal processor, application specific integrated circuit, digital circuit, analog circuit, combinations thereof or other now known or later developed processor. The processor 18 is operable to determine a position of a region of interest within multiple images. For example, the processor 18 is operable to determine the position of a tissue object region of interest in subsequent and/or preceding images within a sequence of images. In one embodiment, the processor 18 is operable to identify an initial region of interest, such as through application of thresholds, gradient processing, or other processes for identifying a desired region within an image. Alternatively, the initial region of interest is identified by the processor 18 in response to user input from the user interface 22.

In one embodiment, the processor 18 is operable to identify regions of interest within a plurality of different images as a function of velocity information. For example, different positions of a region of interest in different images are determined as a function of different velocity estimates for respective ones of the plurality of images. The velocity of an object in one image is used with the time between image acquisitions to estimate a position of the region of interest of the associated object in a subsequent or earlier image. The velocity information used is from a current image or the image to where the region is mapped. Alternatively, the velocity from other images is used in combination or exclusively. In one embodiment, an average velocity associated with a region of interest is used to determine the position of the region. In an alternative embodiment, the region of interest is divided into sub-regions, and each of the sub-regions is separately positioned using the velocity information to form a region of interest with the same or different shape as the region of interest in different images.

In an additional or alternative embodiment, the processor 18 receives physiological cycle signals, such as an ECG signal, for determining a position of a region of interest in different images or assisting in a search. For example, the ECG signal is used to identify a common time through multiple cycles, such as the R wave point, for positioning the region of interest at a same spatial location periodically. As another example, the physiological cycle signal information is used for estimating or modeling behavior of the imaged object within a region of interest. The modeled behavior is then used to determine the position of the region of interest within the different images. Alternatively, the modeled behavior is used to bias other position detection or tracking algorithms. In yet another embodiment, the modeled information is used to limit or assist in search patterns for tracking the region of interest to different positions in different images.

In yet another embodiment, the processor 18 is operable to determine the position of a region of interest using correlation of speckle or a feature, sum of absolute differences, minimum sum of absolute differences, the sum of the squares of the difference or other cost functions. For example, any of the processes and/or associated hardware disclosed in U.S. Pat. Nos. 6,193,660, and 6,527,717, the disclosures of which are incorporated herein by reference, are used.

The input 22 is one or both of a user interface or a source of physiological signals. For example, the input 22 is a keyboard, track ball, mouse, buttons, sliders, touch sensor pads, combinations thereof or other now known or later developed user input devices. In one embodiment, the input 22 is used as a user interface for identification of a region of interest, such as a tissue object region of interest, in one or more but not all of the images of a sequence of images. For example, the user indicates one or more points associated with an object of interest, and the processor 18 generates an outline corresponding to the points using threshold, gradient processes or other algorithms for identifying the region. In yet another example, the user traces an outline of a region of interest. The region of interest information is then stored in a memory, such as the memory 16, for use by the processor 18 to track and other images. As a source of physiological signals, the input 22 is an ECG monitor. Alternatively, a breathing monitor is used. Signals output by the input 22 as a physiological cycle signal is a signal showing the sensed or measured quantity at a given time or a signal derived from cycle information.

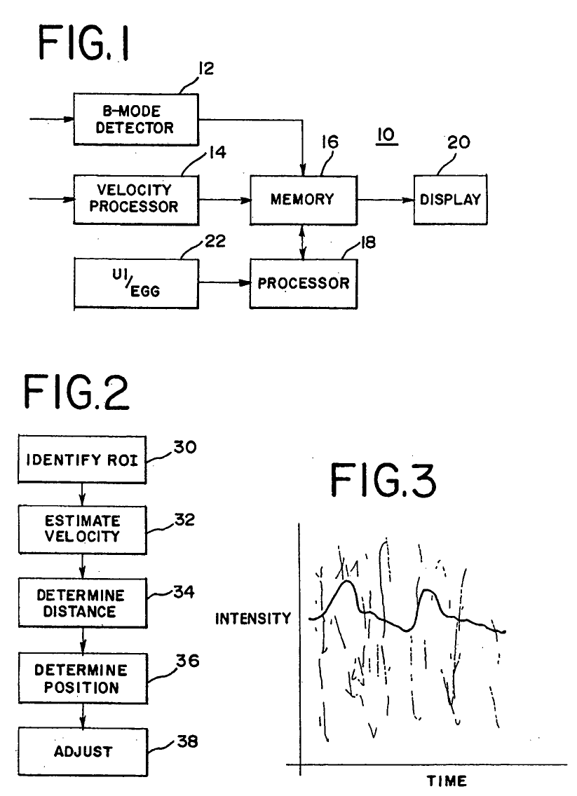

FIG. 2 shows one embodiment of a method for tracking motion of a region of interest, such as a tissue object region of interest, in medical imaging. The method is implemented using the system described above for FIG. 1 or a different system. Different, additional or fewer acts may be provided in the same or different order, such as providing acts 30 through 36 without act 38.

In act 30, a region of interest is identified. In one embodiment, a tissue object region of interest is identified. In alternative embodiments, a fluid region of interest is identified. The region of interest is identified in one or more images. For example, a tissue object region of interest is identified in a multi-dimensional image, such as 2 or 3 dimensional image. As another example, a tissue object region of interest is identified in a portion of an M-mode image. Different one dimensional portions of an M-mode image correspond to a one dimensional image at different times.

The region of interest is identified automatically using a processor or in response to user input. For example, a user selects a point or plurality of points in an M-mode image at a given time as the region of interest. As another example, the user inputs one or more points associated with a region of interest and the processor automatically interconnects the points with either direct lines or curve fitting. User tracing may be used. In yet another alternative embodiment, the processor applies an algorithm to automatically identify a region of interest within an image.

Any image within a sequence of images may be used for initially identifying a region of interest. For example, an original or firstly acquired image is selected and used to identify a region of interest. In an alternative, a subsequent image, such as a lastly acquired image or any image within the sequence, is selected for identifying a region of interest. In yet other embodiments, more than one image is selected for identifying the region of interest. For M-mode images, a one dimensional portion of the image corresponding to the first time in the sequence or a subsequent time in the sequence is selected for identifying the region of interest. The selection is performed automatically by the system or manually by the user. Terms first, second or other numerical designations used herein may distinguish between different images that are or are not the firstly or secondly acquired images within a sequence.

In act 32, a velocity for at least one location associated with a region of interest is estimated. The velocity estimated is a velocity of an object (e.g., fluid and/or tissue) within the region of interest, along the region of interest or at a location relative to the region of interest. In one embodiment, velocities for each spatial location within the region of interest are estimated. A velocity for the region of interest is then calculated from the plurality of estimates, such as through an average or weighted average. Subsets or a single estimate of velocity associated with the region of interest is acquired in other embodiments.

The velocity estimate is of a velocity for the image or of an object. For example, a Doppler velocity estimate is acquired for a tissue object region of interest. Flow or fluid Doppler velocities may be estimated in other embodiments. Doppler velocity is estimated using multiple transmissions in rapid succession to estimate a motion or velocity at a given time of an object. This estimated velocity is not a velocity between images, such as associated with multiple transmissions over a long period of time to allow for a multi-dimensional scan. In alternative embodiments, transmissions associated with different images are used to estimate velocity of the object. For a Doppler estimation, the first lag of auto correlation coefficient indicates a phase change. The phase change is used to calculate velocity. Alternatively, other auto-correlation or temporal correlation functions may be used to estimate the velocity.

Using ultrasound transmissions along scan lines, the estimated velocity is a one-dimensional velocity or a velocity along the scan line direction. For example, a positive valued velocity is associated with movement towards or away from the transducer, and a negative valued velocity is associated with movement the other of away or towards the transducer. Where the actual velocity is along a non-zero degree angle to the scan line, the estimated velocity represents a component of the true velocity. In alternative embodiments, the direction of movement is determined for obtaining a velocity vector, such as a two-dimensional or three-dimensional velocity vector as the estimated velocity. Where the direction of motion is likely along a scan line direction, such as for apical views of the heart, a one-dimensional velocity estimate along the scan line direction may be sufficient. Where the velocity is at an angle to the scan line, a one-dimensional estimate may still be useful.

In act 34, a distance is determined as a function of the velocity estimate. To track the motion or movement of a region of interest between images, the velocity information is used to determine a distance of motion of the region of interest between images based on the rate of motion of a region of interest at a given time. Alternatively, the rate of motion of the region of interest at different times, such as associated with each of the two images, is used to determine the distance of travel of the region of interest from one image to the other image. For one-dimensional motion tracking, such as associated with tracking motion along a scan line direction, the distance is equal to the velocity times the amount of time between images. For example, the motion along the axial direction is determined for M-mode imaging. The velocity at each point within a region of interest is multiplied by the time to the next one-dimensional portion of the M-mode image to determine the distance.

The distance is determined for sequentially adjacent images in a forward or backward direction within a sequence, but may be determined over non-sequentially adjacent images. A new velocity estimate or estimates is used for determination of the distance of movement of the region of interest between each set of sequential images since the velocity may change as a function of time.

For determining a non-axial distance, the direction of motion associated with the velocity estimate is determined, such as using a two-dimensional or three-dimensional velocity vector in response to user input or processor calculation. The direction is then independent of the scan line direction. The velocity is angle corrected as a function of the direction of motion, such as angle correcting a one-dimensional velocity as a function of an angle away from the one dimension.

In an alternative embodiment, two-dimensional tracking is performed using correlation of information between the two images, such as disclosed in U.S. Pat. Nos. 6,193,660, 6,201,900 or 6,527,717, the disclosures of which are incorporated herein by reference. Correlation of at least one portion of an image, such as region of interest, with another potion of a different image, such as a search region of a different image, is performed using a cross-correlation function, correlation function, minimum sum of absolute differences, the squares of differences or other now known or later developed cost function. The velocity information is used to determine an axial distance between images, and the correlation information is used to determine a direction and longitudinal distance. Alternatively, the correlation is used to determine a distance and direction where the distance along the axial direction is a function of both the correlation and the velocity information. Alternatively, the velocity information is used to assist in the correlation search, such as using the velocity to indicate an initial search position, an initial search direction, an initial range of course or fine searches, or other searching parameter for correlating a region of interest of one image with data of a different image. The velocity information may also be used to facilitate axial tracking for three-dimensional regions of interest. Other now known or later developed methods for tracking data may be used in combination with velocity tracking along the axial direction.

In act 36, a position of a region of interest is determined as a function of the velocity using the distance information. For example in an M-mode image, the distance of a region of interest along the axial dimension from one image to a different image is used to determine the position of a point or region of interest in the different image. Where the distance is calculated in terms of the velocity temporal frame of reference, the distance and the amount of time between images in relation to the velocity temporal frame is used to determine the position. The position may be determined for directions other than along the scan line or axially. For example, two- and three-dimensional motion tracking determine the position of a region of interest as a function of velocity with or without correlation. For subsequent or preceding images, the region of interest is then tracked from the current or most temporally adjacent image for which the region of interest position is known.

The position of the region of interest is used for performing quantifications, indicating the region of interest on an image displayed to the user, combinations thereof or for other purposes. FIG. 3 shows one embodiment of indicating the position of the region of interest as tracked through a sequence of images in an M-mode image. Each M-mode image is a copy of the previous image with an additional one-dimensional portion added for further temporal acquisition. The M-mode image is stored as a plurality of one dimensional portions, an entire image, or overlapping M-mode images. As shown in FIG. 3, a tracked region of interest corresponds to a line that varies as a function of time. The user selects a particular point, such as a tissue layer, at a given time. The position of the region of interest or point is tracked in a forward and backwards temporal direction to indicate movement of the tissue as a function of time. For two and three dimensional imaging, the entire region of interest is tracked as a function of time, and the position of the region of interest is indicated with an outline of the region of interest. In one embodiment, the nodes or only a portion of a region of interest is tracked. The tracked portion is then used to define a region of interest in the subsequent image, such as tracking nodes to be used as inputs for determining a region of interest based on the inputs.

As an alternative or in addition to indicating a region of interest throughout the sequence of images on the display, the tracked region of interest is used for quantification. For example, a strain rate or spatial change in velocity due to expansion and contraction is determined. The slope of velocity over a given distance provides the strain rate. An image reflecting strain rate or values reflecting strain rate are provided to the user. Velocity, strain, displacement, volume, or other now known or later developed quantities determined as a function as a region of interest may be determined. Any of various imaging may also be used, such as contrast agent, harmonic, strain rate, Doppler velocity, b-mode, combinations thereof or other now known or later developed imaging modes.

In act 38, the region of interest position is adjusted. In one embodiment, the region of interest is adjusted after determination of a tracked position. Alternatively, the region of interest is adjusted by providing additional initial inputs. Combinations of both may be provided. In one embodiment, the same region of interest is provided in two or more different images within a sequence for determining a region of interests in other images of the sequence. The position for a given image within the sequence is a function of the identified region of interest in the two or more other images.

A region of interest is input for two images of a sequence. For example, the user specifies a region of interest on two or more images, such as image frames 20 and 60 out of 100 frames within a sequence. The region of interest is tracked throughout the sequence using one of the identified regions of interest. The region of interest is tracked in a forward and backward direction. For example, the region of interest designated in frame 20 is tracked forwards and backwards. At frame 60, the user notices an error in the region of interest and adjusts the region of interest. The system then transitions the adjustment throughout the sequence. The adjusted region of frame 60 is then tracked forwards and backwards throughout the sequence. For frames 1 through 20, the region of interest from frame 20 is used for tracking. For frames 21 through 59, a weighted average of the position determined from the region of interest of frame 20 and frame 60 is performed. The weights vary as a function of the temporal relationship of a given frame to frames 20 and 60. For example, the position from frame 20 is more heavily weighted for frame 21 than the position from frame 60. A linear or nonlinear change in weighting may be provided. For frames 61 through 100, the region of interest tracked from frame 60 is used. In alternative embodiments, different weighting schemes, different combination schemes and application of the combination to different frames are provided, such as providing a weighted combination of a region of interest tracked from both frames 20 and 60 for frames 1 through 20 and 60 through 100. Other combinations then averaging or weighted averaging may be used, such as a non-linear look up table.

In another embodiment allowing adjustment of the region of interest, the region of interest is modified so that the same position is provided for the region of interest in images representing the same portion of a physiological cycle. For example, the region of interest is positioned at a same location within the image along at least one or all dimensions for a same portion of an ECG wave, such as the R wave. The physiological cycle is tracked. The estimation of velocity and determination of the position for each of a plurality of images within the sequence corresponding to one or more physiological cycles is repeated. The position determined for at least some of the repeats is determined as a function of an expected position at the time of the physiological cycle. Using the example above, the 20^{th} and 60^{th} frames of data correspond to a same portion of a physiological cycle. A region of interest is identified for frame 20 or identified for a different frame and tracked to frame 20. An adjustment for frame 60 so that the region of interest of frames 20 and 60 matches is determined. The adjusted position for frame 60 is then used as discussed above to track the region of interest in combination with the region of interest identified for frame 20 or a different frame.

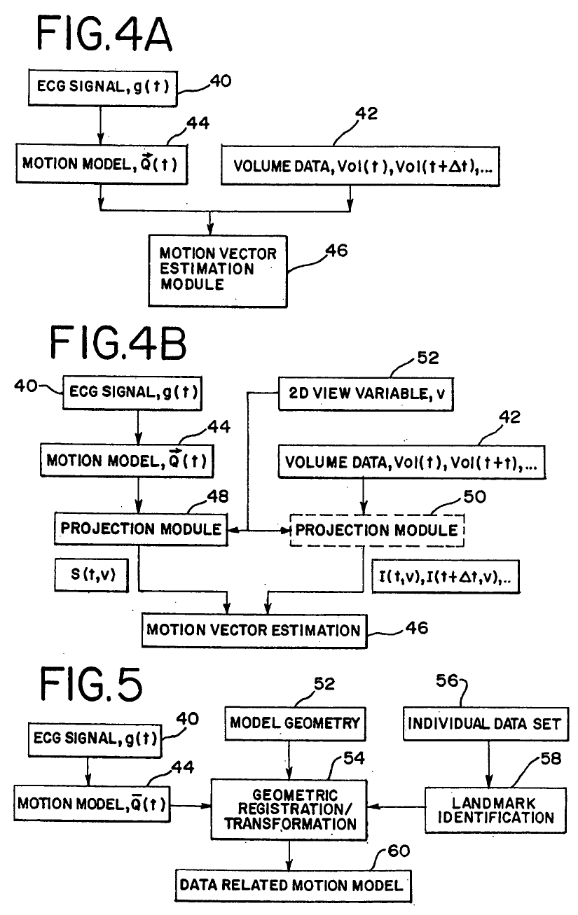

FIGS. 4A, 4B and 5-7 are flow charts representing different embodiments of methods for tracking a region of interest in medical imaging, such as ultrasound imaging. These embodiments are used alone or in any possible combination with each other and/or the velocity tracking discussed above. The system of FIG. 1 or a different system is used. Different, additional or fewer acts may be provided in the same or different order for any of the methods. Modeling of the region of interest is used to assist in motion tracking.

Referring to FIG. 4A, a region of interest is identified in at least one image of a sequence of images, such as the above discussed act 30. Additional information may also be provided, such as the type of region of interest identified. For example, the user configures an imaging system for a particular type of imaging (e.g., apical view cardiac imaging), selects a list of types of imaging, or otherwise indicates the organ, tissue, fluid or combinations thereof being imaged.

In act 40, a physiological cycle signal is obtained. Any physiological cycle signal may be used, such as an ECG or breathing cycle signal. The ECG signal and associated heart cycle is discussed below, but other physiological cycles and signals may be used. The signal is obtained from a sensor separate from an imaging system, such as an ECG sensor. Alternatively, the imaging system processes ultrasound data (e.g., velocity or variance data) as a function of time to determine the physiological cycle signal. The signal represents the cycle as a function of time, such as an intensity or other characteristic of the cycle as a function of time. Alternatively, the signal is derived from cycle information, such as being a time period, frequency, range or other parameter of a cycle. While the preceding description describes the general use of the ECG signal, the analysis can be simplified by using only a single metric derived from the ECG signal, such as the elapsed time from the preceding R-wave, which is a component of the ECG signal. The motion model may then incorporate spatially varying estimates of the cardiac motion for different points in time based on the time from the previous R-wave event. The model may also include the time between consecutive R-waves, and the model can be scaled.

The physiological cycle signal is used as one source of data to assist in tracking. Two different sensor types are used. The information from the two different data collection systems are combined to improve displacement or motion detection, whether the displacement is used for tracking or registration or another purpose. Data processing transforms the two different system signals to actuate interconnected discrete hardware elements. For example, to utilize specialized hardware for ECG detection and cardiac cycle processing while utilizing other specialized hardware and software for image motion tracking, the image motion tracking algorithm uses input from an ECG module as a parameter in the motion tracking algorithm. In another example in ultrasound, velocity estimates, such as from a Doppler processor, are used to detect tissue motion while utilizing echo processing hardware for improved image quality and using the velocity estimate as input into a motion tracking or image registration algorithm on echo image data in conjunction with ECG data. While these processes are described as hardware modules, they can also be implemented in software running on general purpose CPUs or special purpose microchips, including, but not limited to, DSP chips.

The position of the region of interest in different images is determined as a function of estimates from different sources, such as ECG information and velocity or correlation tracking. In act 42, the position of the region of interest is tracked as discussed above or using correlation. For example, an estimate of the position of a region of interest in one image relative to another image is determined as a function of ultrasound data. In one embodiment using correlation, a portion of the image, such as the identified region of interest, is matched with a portion of the other image. In another embodiment, velocity estimates with or without correlation matching between images are used to determine the position. Other types of data may be used alone or in combination.

The second type of data for determining the position of the region of interest is the physiological cycle signal. The position is determined as a function of both the matching and the physiological cycle signal. For example, different positions determined from different types of data are averaged or otherwise combined. As another example, the match using one type of data (e.g., ultrasound) is calculated as a function of a correlation between portions of different images and a deviation from a modeled motion using another type of data, such as the ECG signal. Improvements in motion tracking and analysis can be obtained by incorporating the ECG or other physiological signal, such as in heart motion analysis. Such improvement can be achieved by mathematically modeling the average cardiac contraction and relaxation for each time point within a cardiac cycle using some closed form functions in act 44. These closed form functions describe or model the spatial locations of the myocardial wall, as a function of time. With the input of the ECG signal to indicate timing information in the cardiac cycle, a probability function, a priori describing the motion tendency, may be employed in the motion estimation algorithm.

A position of the region of interest identified in one image is determined for a different image as a function of the physiological cycle signal. The change in position or absolute position is modeled as a function of a physiological cycle signal. Motion of the region of interest is modeled. Based on the time within the cycle, the position is determined. The model provides an independent estimation of position or is used as a variable in another estimation of position. Using two different estimates or an estimate determined as a function of two different types of data, the position of the region of interest is determined in act 46. Suppose \vec{R}(t)εR³ is the spatial position of a particular very small volume in the organ, vol(t), for instance the heart muscle. The position of one spatial point can be represented as a periodic function, \vec{R}(t+nτ)=\vec{Q}(t), tε[0,τ), where τ is the interval or period of the cardiac cycle. The respiratory motion effect is negligible or accounted for through additional calculations. The reference time, t, is aligned with the ECG signal, or is the output from processing the ECG signal, g(t). The origin of the time axis is a time point within the cardiac cycle, for instance, the time corresponding to R-wave peak. The reference point of \vec{R}(t) can be set as the some landmark point of the organ. The entire region of interest or a manually or automatically selected sub-set (e.g., point) with the region of interest is used as a landmark.

In the two-dimension application represented in FIG. 4B, both \vec{R}(t) and vol(t) are projected in acts 48 and 50 onto the imaging or scan plane, denoted as \vec{S}(t, v) and I(t, v), where v is the projection variable 52 corresponding to a different view of the two-dimension image formation. v corresponds to a plane rather than a single variable. Alternatively, a single variable is used to represent a standard imaging plane, such as the parastemal short-axis view, the apical long-axis view, and so on.

Referring to FIGS. 4A and 4B, the construction of the motion model used in act 44 is obtained from mathematically modeling the organ dynamics or fitting an appropriate function using manual analysis data for the cardiac cycle. When formulating the algorithm, the velocity, \vec{U}(t)=[Uₓ(t),U_{y}(t),U_{z}(t)]^{T}, is used rather than the spatial position \vec{Q}(t), but the relationship

$$\vec{U} ( t ) = \frac{ⅆ \vec{Q} ( t )}{ⅆ t}$$

allows derivation of the position information from velocity information. Considering the variations of the velocity in different subjects, a statistical model may be used rather than a fixed model for each instant time. Therefore, for each component of \vec{U}(t), there is an associated probability density function. Denoting the probability functions as P(u_{x,}t), P(u_{y,}t), and P(u_{z,}t), the model parameters are the means \vec{U}(t)=[\overline{U}ₓ(t),\overline{U}_{y}(t),\overline{U}_{z}(t)]^{T}, and the standard deviations, \vec{σ}(t)=[σₓ(t),σ_{y}(t),σ_{z}(t)]^{T}. The means and deviations are functions of the spatial variables x, y, and z. To reduce the dimensionality of the data set, principle component analysis (PCA) can be used to model the myocardial wall. These principle components form a shape space. Using a Kalman-filter-based technique, the myocardial wall can be tracked using shape space and space transition equations. Instead of being constant, each element in the transition matrix of the space transition equation can be a function of time.

In an alternative organ model, the prolate spheroidal coordinate system is used. For instance in heart wall description, a parametric surface description is used. Denoting the parameters as u and v, the three prolate spheroidal coordinates are functions: ξ=ƒ₁(u, v),η=ƒ₂(u, v), and φ=ƒ₃(u, v), with a u-v plane corresponding to the unfolded heart wall muscle sheet, and {ξ,η,φ} representing the spatial location of the myocardial wall. The combination of information from acts 42 and 44 to estimate the motion vector or position between images is shown in FIGS. 4A and 4B. FIG. 4A shows the fusion of volume data and the ECG signal. FIG. 4B shows the fusion of the image data and ECG signal. The fusion of a priori information into the image data or motion estimation algorithm is through the addition of an inertia term in the motion vector computation in one embodiment. Motion vector estimation determines a sufficient or best match of the data and is derived from a position \vec{R}(t) on a first image to a position \vec{R}(t+Δt) on a second. For example, the match is mathematically represented as:

$${MIN}_{{[ {\hat{u}}_{x} , {\hat{u}}_{y} , {\hat{u}}_{z} ]}^{T} ∈ [ - {\vec{U}}_{N} , {\vec{U}}_{P} ]} \{ F ( vol ( t ) , vol ( t + Δ t ) , {\hat{u}}_{x} , {\hat{u}}_{y} , {\hat{u}}_{z} ) \} \qquad ( 1 )$$

where {ûₓ,û_{y},û_{z}} are estimated velocities in x, y, and z directions, and F(·) can be a sum of absolute difference (SAD), sum of squared difference (SSD), other forms discussed herein or as used in optical flow which makes use of the gradients of the data as well.

The search for a match between images may be unconstraining in one embodiment of motion estimation. In another embodiment, \vec{U}_{N}, and \vec{U}_{P} are the maximum velocity bounds in the negative axes and positive axes. \vec{U}_{N}, and \vec{U}_{P} are fixed or are periodic as a function of time.

The predicted or modeled motion or position is incorporated into the representation of equation (1). The equation is modified to become:

$${MIN}_{{[ {\hat{u}}_{x} , {\hat{u}}_{y} , {\hat{u}}_{z} ]}^{T} ∈ [ - {\vec{U}}_{N} , {\vec{U}}_{P} ]} \{ F ( vol ( t ) , vol ( t + Δ t ) , {\hat{u}}_{x} , {\hat{u}}_{y} , {\hat{u}}_{z} , P ( {u}_{x} , t ) , P ( {u}_{y} , t ) , P ( {u}_{z} , t ) \} \qquad ( 2 )$$

In equation (2), the SAD and SSD methods may be used with an additional term which measures the error between the estimated matching motion vector {ûₓ,û_{y},û_{z}} and the motion vector [\overline{U}ₓ(t),\overline{U}_{y}(t),\overline{U}_{z}(t)]^{T} provided by the model. Using the exponential form of the probability function as an example, the following equation can be applied for the motion vector estimation in combination with a SAD matching method:

$$F ( \cdot ) = \sum \vert vol ( x , y , z ) - vol ( x + {\hat{u}}_{x} Δ t , y + {\hat{u}}_{y} Δ t , z + {\hat{u}}_{x} Δ t ) \vert - \\ exp ( - \frac{{( {\hat{u}}_{x} - {\overline{U}}_{x} )}^{2}}{{σ}_{x}^{2}} - \frac{{( {\hat{u}}_{y} - {\overline{U}}_{y} )}^{2}}{{σ}_{y}^{2}} - \frac{{( {\hat{u}}_{z} - {\overline{U}}_{z} )}^{2}}{{σ}_{z}^{2}} ) \qquad ( 3 )$$

In this equation, the exponential term accounts for the physiological cycle signal and responsive modeling parameters. Other forms of the density functions and other methods to incorporate the density function in F(.) may be used.

In a further embodiment, the model is adjusted as a function of the region of interest. For example, the location and motion of a region of interest for one patient is different than for another patient, because of differences in the overall volume, strain, flexibility, muscle thickness or other parameter. The model is altered to account for one or more of these possible differences. The imaging system automatically determines the parameter for a given patient or the user inputs the parameter value. The model then estimates a position or motion vector as a function of the parameter. Motion vector and position may be used interchangeably as the motion vector is used to identify the position and the difference in positions provides the motion vector.

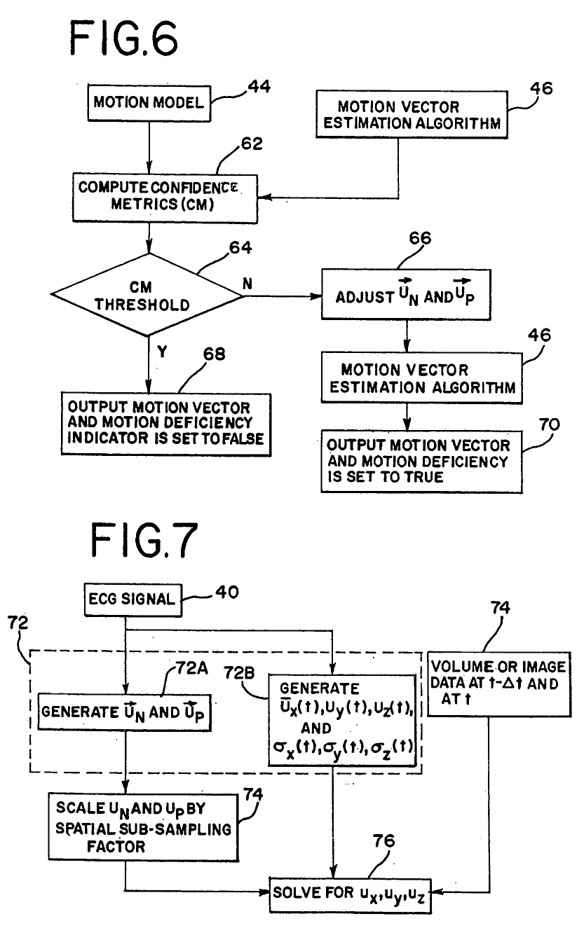

FIG. 5 represents adjusting the model as a function of the patient being scanned. A generic model is provided in act 52. In the motion estimation module, the generic model geometry is mapped to a data related motion model based on the individual input data in act 54. In the generic model description, the maximum dimension of the organ is normalized to 1.0. Given a data set from a specific subject in act 56, a geometrical registration and transformation process is used to register the model spatial dimension to fit the individual data set at some predetermined time point, e.g., to fit the model surface to the endocardium of the cardiac ventricle at the end diastole time point in act 54. This registration can be done by first identifying the landmarks in the organs automatically or manually in act 58. The landmark points are then used as the reference points in the registration process. This registration is only required one time, but may be used multiple times for a same sequence of images. The data based model geometry at other points in the cycle is controlled by the motion model functions. A data related motion model is provided in act 60.

As an alternative or in addition to using the model and physiological cycle signal for estimating position, the model and physiological cycle signal are used to assist in or speed correlation or matching searches. For example, the physiological cycle signal is used to limit a search region of the matching operation where the limits are a function of the physiological signal. As another example, a search for a sufficient match is guided as a function of the physiological signal, such as identifying a beginning search location, a search step size, a search resolution, a search direction, and/or combinations thereof based on a model.

In one embodiment, the ECG signal indicates the phase of the contraction and relaxation of the heart and motion of organs associated with cardiac motion. The maximum of the velocity or change in position at each instant time may be associated with the ECG signal. Rather than fixing the search region of the motion estimation, such as represented by equations (1) and (2), \vec{U}_{N}, and \vec{U}_{P} may be periodic functions of time. At different instants in time, the velocity estimation solvers use different velocity bounds based on the model and physiological cycle signal, resulting in more efficient computations or improving the computation speed.

FIG. 6 shows one embodiment of a method for limiting a search as a function of a physiological cycle signal. The model prediction is used in two paths. In one path represented by act 46, the model is used in calculation of the motion vector as described in other paragraphs in this document as well as in equations (1) through (3). In another path, the model provided in act 44 is used to estimate the deficiency of the motion in act 62 and to initiate an additional search direction for the motion vector by adjusting the maximum velocity bounds \vec{U}_{N}, and \vec{U}_{P} in act 66. For example, confidence metrics are computed in act 62 by measuring the agreement between the predicted value given by the motion model and the result given by the motion estimation algorithm. If the confidence metrics indicate a sufficient agreement between the modeled position and the matched position, the motion vector or position is output while setting a deficiency indicator to false in act 68. If the confidence metrics indicate an insufficient agreement, the limits of the matching are adjusted, allowing a greater search region. The motion vector or position is then recalculated in act 46 and a motion vector associated with the best match is output in act 70. The motion deficiency flag is set to true to indicate the match may be deficient.

In another embodiment represented in FIG. 7, the physiological cycle signal is used to guide the search for a sufficient match of a position or motion vector through coarse and then fine searching. The motion vector is the product of the velocity vector and the volume (or image) sampling interval. In many applications, the sampling strategy is even sampling in time. The motion vector estimation is obtained once the velocity vector is determined. In act 72, the limits of the search are calculated from the model (act 72A) and the estimated velocity vector or position and standard deviation as calculated (act 72B) as a function of the physiological cycle signal. The medical data representing at least two different scans at different times (e.g., different images of the same region) is acquired in act 74. In act 76, the search limits are scaled by sub-sampling in any of various resolutions. Act 76 is repeated at different resolutions based on the limits. For example, the algorithm is carried out in coarse to fine strategy. In a cardiac wall motion analysis application, the myocardial wall is represented using a mesh. The velocity computation is first carried out in a mesh with sparse vertices and gradually in one with dense vertices based on the matches in with the sparse vertices. This method may improve the computation speed. For each resolution, the velocity vector estimation is performed in act 76, such as described above using the equations (1), (2) or (3).

In a further additional or alternative embodiment, the position determined for the region of interest is biased to be at a same location for images within the sequence of images for a same portion of a physiological cycle. Using the physiological cycle signal, the images associated with the same positioning are identified. The tracking is constrained to return to the same location for subsequent cardiac cycles. This constraint may be absolute, or the motion estimation cost function may impose additional cost for a tracking that deviates from the same spatial location on repeated cardiac cycles. This additional constraint can be applied to all frames where tracking is applied, or applied to a limited number of frames, such as the first frame after each R-wave.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A method for tracking motion of a tissue object region of interest in medical imaging, the method comprising:
(a) identifying the tissue object region of interest in a first image;
(b) estimating a velocity for at least one location associated with the tissue object region of interest; and
(c) determining a position of the tissue object region of interest in a second image as a function of the velocity, the first image acquired at a different time than the second image.

2. The method of claim 1 wherein (a) comprises receiving a user selected indication of the tissue object region of interest.

3. The method of claim 1 wherein (b) comprises obtaining a tissue Doppler velocity estimate of a velocity of the object.

4. The method of claim 1 further comprising:
(d) determining a distance as a function of the velocity;
wherein (c) comprises determining the position as a function of the distance and an amount of time between the first image and the second image.

5. The method of claim 1 further comprising:
(d) indicating the position on the second image.

6. The method of claim 1 wherein the first image comprises a first one dimensional portion of an M-mode image and the second image comprises a second one dimensional portion of the M-mode image, the first portion associated with a different time than the second portion on the m-mode image.

7. The method of claim 1 wherein (b) comprises estimating the velocity as a one-dimensional estimate along at least one scan line.

8. The method of claim 7 further comprising:
(d) determining a direction of motion associated with the velocity, the direction independent of a scan line direction; and
(e) angle correcting the velocity as a function of the direction.

9. The method of claim 1 wherein the first and second images are multi-dimensional images;
further comprising:
(d) correlating at least a portion of the first image with at least a portion of the second image;
wherein (c) comprises determining the position as a function of the velocity and the correlation.

10. The method of claim 1 further comprising:
(d) tracking a physiological cycle;
(e) repeating (b) and (c) for each of a plurality of images within the physiological cycle, the plurality of images including the second image;
(f) adjusting the position determined for at least some of the repeats of (c) pursuant to (e) as a function of an expected position at a time in the physiological cycle.

11. The method of claim 1 wherein the first and second images comprise two images of a plurality of stored images;
further comprising:
(d) identifying the tissue object region of interest in a third image; and
(e) adjusting the position as a function of the identified tissue object region of interest in the third image.

12. A system for tracking motion of a tissue object region of interest in medical imaging, the system comprising:
a display operable to display a sequence of images;
a memory operable to store an identification of the tissue object region of interest in a first image of the sequence of images;
a velocity estimate processor operable to estimate a velocity for at least one location associated with the tissue object region of interest; and
a further processor operable to determine a position of the tissue object region of interest in a second image of the sequence of images as a function of the velocity, the first image acquired at a different time than the second image.

13. The system of claim 12 further comprising a user interface, wherein the identification of the tissue object region of interest in the first image is responsive to input from the user interface.

14. The system of claim 12 wherein the display is operable to indicate the position of the tissue object region of interest in a plurality of images of the sequence of images including the second image and wherein the further processor is operable to determine different positions as a function of different velocity estimates for respective ones of the plurality of images.

15. The system of claim 12 wherein the sequence of images comprise at least one of: (i) temporally different portions of an M-mode image, (ii) a set of two-dimensional B-mode images, (iii) a set of two-dimensional Doppler images, (iv) a set of three dimensional representations or (v) combinations thereof.

16. A method for tracking a region of interest in medical imaging, the method comprising:
(a) identifying the region of interest in a first image;
(b) obtaining a physiological cycle signal; and
(c) determining a first position of the region of interest in a second image as a function of the physiological cycle signal, the second image different than the first image.

17. The method of claim 16 wherein (c) comprises modeling motion of the region of interest, the first position being a function of the model.

18. The method of claim 17 further comprising:
(d) adjusting a model used in (c) as a function of the region of interest.

19. The method of claim 16 further comprising:
(d) matching a first portion of the first image with a second portion of the second image;
wherein (c) comprises determining the first position as a function of both the matching and the physiological cycle signal.

20. The method of claim 19 further comprising:
(e) modeling motion of the region of interest from the first image to the second image as a function of the physiological cycle signal;
wherein (c) comprises determining the first position as a function of both the matching and the modeling.

21. The method of claim 20 wherein (c) and (d) comprise determining a first match as a function of correlation of the first portion to the second portion and a deviation from the modeled motion.

22. The method of claim 16 wherein (b) comprises receiving an ECG signal.

23. The method of claim 16 further comprising:
(d) matching a first portion of the first image with a second portion of the second image;
wherein (c) comprises limiting a search region of the matching of (d) as a function of the physiological signal.

24. The method of claim 16 further comprising:
(d) matching a first portion of the first image with a second portion of the second image;
wherein (c) comprises guiding a search for a sufficient match of (d) as a function of the physiological signal.

25. The method of claim 16 wherein (c) comprises biasing the first position to be at a same location for images within the sequence of images for a same portion of a physiological cycle associated with the physiological cycle signal.

26. A method for tracking a region of interest in medical imaging, the method comprising:
(a) obtaining a first estimate of a position of a region of interest in a first image relative to a second image as a function of a first type of data;
(b) obtaining a second estimate of the position of the region of interest in the first image relative to the second image as a function of a second type of data different than the first type of data; and
(c) identifying the position as a function of the first and second estimates.

27. The method of claim 26 wherein (a) comprises matching a first portion of the first image with a second portion of the second image, the first and second images being responsive to ultrasound data, and wherein (b) comprises modeling a change in position as a function of a physiological cycle signal.

28. The method of claim 26 wherein (a) comprises matching a first portion of the first image with a second portion of the second image, the first and second images being responsive to ultrasound data, and wherein (b) comprises obtaining the second estimate as a function of a velocity estimate.

29. The method of claim 1 wherein (b) comprises estimating the velocity for each of a plurality of sub-regions of the tissue object region of interest; and
further comprising:
(d) modifying a shape of the tissue object region of interest as a function of differences in the velocity for each of the plurality of sub-regions.
